(19) United States
(12) Patent Application Publication
(10) Pub. No.: US 2021/0236191 A1
(43) Pub. Date: Aug. 5, 2021

(54) RF ABLATION CANNULA WITH INJECTION PORT

(71) Applicant: BOSTON SCIENTIFIC NEUROMODULATION CORPORATION, Valenvia, CA (US)

(72) Inventors: Kevin P. Wang, Fremont, CA (US); Frank Louro, Mission Viejo, CA (US)

(21) Appl. No.: 17/165,502

(22) Filed: Feb. 2, 2021

Related U.S. Application Data
(60) Provisional application No. 62/969,969, filed on Feb. 4, 2020.

(51) Int. Cl. A61B 18/14

(52) U.S. Cl. CPC A61B 18/14; A61B 2018/00577

(57) ABSTRACT

In some aspects, the present disclosure pertains to RF ablation cannulas that comprise: a cannula shaft and a cannula hub that comprises an insertion port that is configured to receive an electrode shaft, a stylet shaft, or both and an injection port comprising a mating feature that is configured to interface with an injection device, and seal that is configured to form a water-tight seal around the shaft when it is inserted through the insertion port. By providing a water-tight seal around the shaft, backflow of pressured fluid around the shaft (e.g., backflow of pressurized fluid that is injected into the injection port) is prevented. Other aspects of the present disclosure pertain to systems that comprise such RF cannulas and to methods of using such systems.

### CROSS-REFERENCE TO RELATED APPLICATIONS

The present application claims the benefit of U.S. Provisional Application Ser. No. 62/969,969, filed Feb. 4, 2020, the disclosure of which is herein incorporated herein by reference in its entirety.

### FIELD OF THE DISCLOSURE

The present disclosure relates to cannulas for use in RF ablation.

### BACKGROUND

The present disclosure relates to RF ablation for pain management in the peripheral nervous system. A traditional RF ablation procedure includes the following steps: (1) injecting a local anesthetic on skin surface near a cannula entry point, (2) inserting a cannula into patient to target location, (3) removing the cannula stylet and inserting an electrode into cannula, (4) performing sensory and motor testing with electrode inserted in order to verify cannula placement, adjusting placement as necessary, (5) removing the electrode from the cannula, (6) attaching a syringe of local anesthetic to the cannula, (7) injecting local anesthetic at the target location, (8) removing the syringe from the cannula, (9) re-inserting the electrode into the cannula, (10) ablating tissue, and (11) removing the electrode and the cannula from the patient.

The present disclosure provides an improved cannula design and an improved method using the cannula design, in which the number of steps is reduced relative to the above-described traditional RF ablation procedure.

### SUMMARY

In some aspects, the present disclosure pertains to RF ablation cannulas that comprise: (a) a cannula shaft; and (b) a cannula hub that comprises (i) an insertion port that is configured to receive a shaft (e.g., an electrode shaft, a stylet shaft, or both), (ii) an injection port comprising a mating feature that is configured to interface with an injection device, and (iii) seal that is configured to form a water-tight seal around the shaft when the shaft is inserted through the insertion port. By providing a water-tight seal around the shaft, backflow of pressured fluid around the shaft (e.g., backflow of pressurized fluid that is injected into the injection port) is prevented.

In some aspects, the present disclosure pertains to RF ablation cannulas that comprise: (a) a cannula shaft comprising a proximal end, a distal end, and a shaft lumen; and (b) a cannula hub comprising a proximal end, a distal end, a primary lumen having a first axis, a proximal end and a distal end, the primary lumen extending from the proximal end to the distal end of the cannula hub, wherein the distal end of the primary lumen terminates at a proximal end of the shaft lumen and the proximal end of the primary lumen terminates at an insertion port that is configured to receive an electrode shaft, a stylet shaft, or both, a secondary lumen having a proximal end and a distal end, the secondary lumen laterally branching from the primary lumen, wherein the proximal end of the secondary lumen terminates at the primary lumen and the distal end of the secondary lumen terminates at an injection port comprising a mating feature that is configured to interface with an injection device, and a seal that is configured to form a water-tight seal around a shaft that is inserted through the insertion port. By providing a water-tight seal around the shaft, backflow of pressured fluid around the shaft (e.g., backflow of pressurized fluid that is injected into the secondary lumen) is prevented.

In some embodiments, which can be used in conjunction with the above aspects, the seal comprises a proximal component, a distal component, and an elastomeric gasket between the proximal component and the distal component through which a portion of the primary lumen passes, wherein axial movement of the proximal component toward the distal component causes the elastomeric gasket to form the water-tight seal around the shaft that is inserted through the insertion port and wherein axial movement of the proximal component away from the distal component causes the elastomeric to release the shaft that is inserted through the insertion port.

In some embodiments, which can be used in conjunction with any of the above aspects and embodiments, rotation of the proximal component relative to the distal component in a first direction results in the axial movement of the proximal component toward the distal component and wherein rotation of the proximal component relative to the distal component in a second direction opposite the first direction results in the axial movement of the proximal component away from the distal component.

In some embodiments, which can be used in conjunction with any of the above aspects and embodiments, the cannula hub comprises a core component that contains a portion of the primary lumen and at least a portion of the secondary lumen, the secondary lumen laterally branching from the primary lumen within the core component. In some of these embodiments, the distal component may be either attached to the core component or may be integrated with the core component as single unified component.

In some embodiments, which can be used in conjunction with any of the above aspects and embodiments, the seal comprises one or more radially extended features that assist in rotating the proximal component relative to the distal component. In some of these embodiments, the one or more radially extended features actuate the proximal component. For example, the one or more radially extended features may be provided on the proximal component, or the one or more radially extended features may be provided on an adaptor cap that attached to the proximal component.

In some embodiments, which can be used in conjunction with any of the above aspects and embodiments, the mating feature for the injection port may be a luer interface.

In some embodiments, which can be used in conjunction with any of the above aspects and embodiments, the cannula hub comprises a core component that contains a portion of the primary lumen and at least a portion of the secondary lumen, the secondary lumen laterally branching from the primary lumen within the core component, and the core component comprises the injection port. In some of these embodiments, the RF ablation cannula further comprises an injection hub comprising the injection port and a flexible tube connecting the injection hub to the core component, and the secondary lumen extends from the core component, through the flexible tube, through the injection hub and terminates at the injection port.

In some embodiments, which can be used in conjunction with any of the above aspects and embodiments, the cannula shaft comprises a bend proximate a distal end of the cannula shaft and wherein the cannula shaft comprises a sidewall opening proximate the bend.

In other aspects, the present disclosure provides systems that comprise (a) an RF ablation cannula in accordance with any of the above aspects and embodiments and (b) a stylet comprising a stylet hub and a stylet shaft, the stylet shaft being insertable through the insertion port of the cannula hub and into the cannula shaft.

In some embodiments, the cannula shaft has a first bend, the stylet shaft has a second bend, and a longitudinal position of the first bend corresponds to a longitudinal position of the second bend when the stylet may be fully inserted into the RF ablation cannula.

In some embodiments, which can be used in conjunction with any of the above aspects and embodiments, the cannula hub comprises a first feature and the stylet hub comprises a second feature that may be configured to engage with the first feature when the stylet is fully inserted into the RF ablation cannula and the stylet shaft is positioned at a specific predetermined rotational orientation relative to a rotational orientation of the cannula shaft. For example, one of the first and second features may be inserted into the other of the first and second features in order to engage the first and second features. In these embodiments, the seal may comprise a proximal component, a distal component, and an elastomeric gasket between the proximal component and the distal component, the cannula hub may comprise an indexing component rotationally fixed with the distal component while allowing rotation of the proximal component relative to the distal component, and the indexing component may comprises the first feature.

In some embodiments, which can be used in conjunction with any of the above aspects and embodiments, the system further comprises an RF ablation electrode having an electrode shaft.

In still other aspects, the present disclosure provides methods for RF ablation using a system in accordance with any of the above aspects and embodiments. The method comprises: inserting the RF ablation cannula and stylet into a target location in a patient; removing the stylet; inserting the RF ablation electrode into the cannula; tightening the seal onto the electrode shaft; performing sensory and motor testing on the patient with the RF ablation electrode; injecting local anesthetic through the injection port to the target location; and ablating tissue with the RF ablation electrode.

Other aspects and embodiments will become apparent to those of ordinary skill in the art upon review of the detailed description and claims to follow.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1A is an exploded view of an RF ablation system, including an RF ablation cannula and a stylet, in accordance with an embodiment of the present disclosure.

FIG. 1B is an assembled view of the RF ablation system of FIG. 1A.

FIG. 1C is an assembled view of an RF ablation system, including an RF ablation cannula and a stylet, in accordance with an alternative embodiment of the present disclosure.

FIG. 2 is an assembled view of a distal end of an RF ablation system like that of FIG. 1A.

FIG. 3A is a disassembled view of an RF ablation system, including an RF ablation cannula and a stylet, in accordance with another embodiment of the present disclosure.

FIG. 3B is an assembled view of the RF ablation system of FIG. 3A.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Various embodiments according to the present disclosure are described below.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

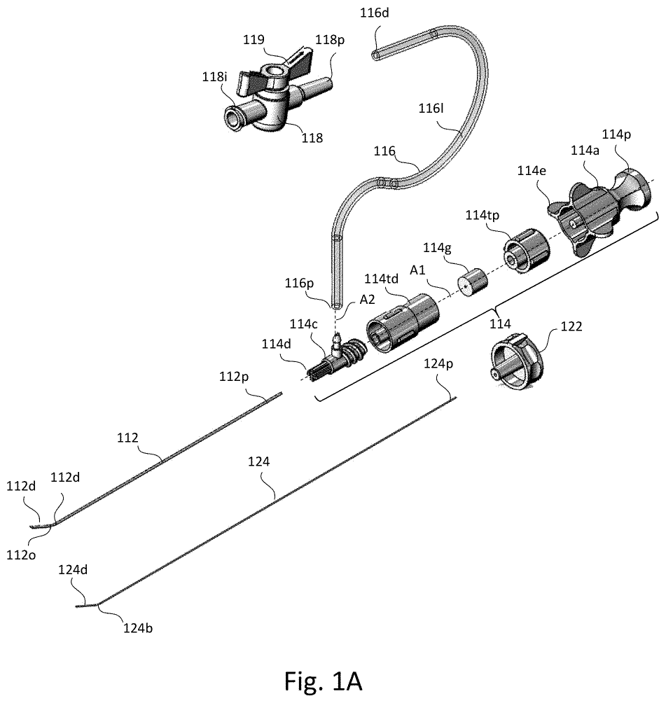

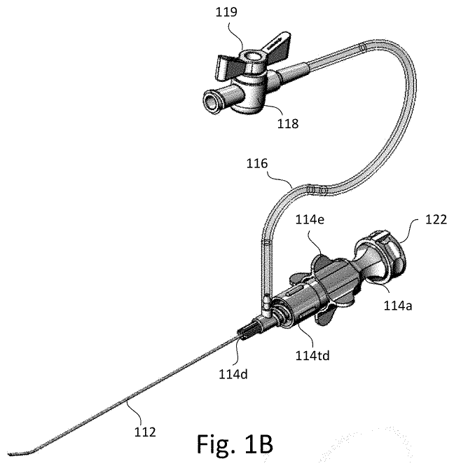

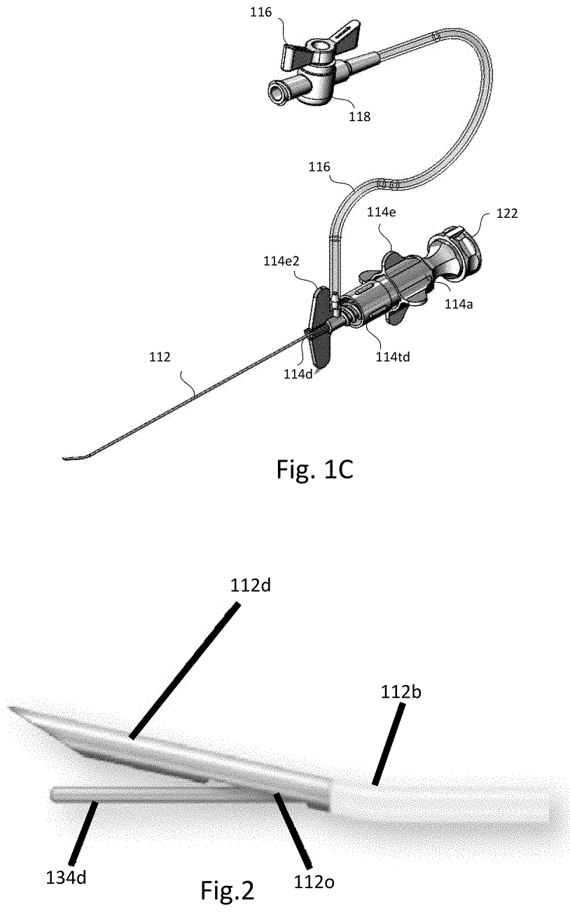

Referring now to FIGS. 1A and 1B, an RF ablation system is shown, which includes an RF ablation cannula and a stylet for use with the RF ablation cannula.

The cannula includes a hollow cannula shaft 112 having a cannula lumen, a proximal end 112p, and a distal end 112d including a sharp tip for penetrating tissue. The cannula shaft 112 may be of an suitable gauge and may range, for example, from gauge 16 to gauge 23.

The cannula further includes a cannula hub 114 having a proximal end 114p and a distal end 114d. The cannula hub 114 comprises a primary lumen having a first axis A1 extending from the proximal end 114p to the distal end 114d of the cannula hub 114, wherein the distal end of the primary lumen (which is at the distal end 114d of the cannula hub 114) terminates at a proximal end of the shaft lumen and is co-linear with the shaft lumen and wherein the proximal end of the primary lumen (which is at the proximal end 114p of the cannula hub 114) is configured to receive a stylet shaft or an electrode shaft and is configured to interface with a stylet hub or an electrode hub. The cannula hub 114 includes a seal that is configured to form a water-tight seal around a stylet shaft or an electrode shaft.

Please note that, as used herein, “proximal end” generally refers to the end of a component that lies closer to an operator holding the cannula hub, and “distal end” generally refers to the end of a device or object that lies further from the cannula hub.

In the embodiment shown, the cannula shaft 112 comprises a bend 112b at the distal end 112d thereof. For example, the cannula shaft 112 may comprise a bend 112b having an angle ranging from 5 to 45 degrees, typically 10 to 20 degrees. In such embodiments, the cannula shaft 112 may be provided with a side opening 1120 proximate the bend 112b, through which an inserted electrode may be guided such that the distal end of the electrode 134d extends out of the side opening 1120 (see FIG. 2). When electrical current is supplied to such an assembly, ablation current flows through a volume larger than would otherwise be affected by an assembly in which the electrode extends out of the distal tip of the cannula shaft 112.

The cannula also comprises a secondary lumen having a proximal end that branches from the primary lumen and a distal end that terminates at an injection port comprising a mating feature that is configured to interface with an injection device.

Referring again to FIGS. 1A-1B the core component 114c of the cannula hub 114 comprises at least a proximal portion of the secondary lumen and has a second axis A2. The secondary lumen branches from the primary lumen at an angle. The angle of the secondary injection lumen is not important but it is preferred to be in a manner that minimizes the tortuousness of the path for the fluid flow during injection. In some embodiments, the angle between the first axis and the second axis may range between 30 and 90 degrees.

In FIGS. 1A-1B, the RF ablation cannula further comprises an injection hub 118 that comprises an injection port 118i having a mating feature that is configured to interface with an injection device and an outlet port. For example, the mating feature may comprise a male or female luer interface (the mating feature is a female luer taper with a female luer twist thread on the exterior wall in the embodiment shown) or any other type of fluid connection. The ablation further comprises a flexible tube 116 a proximal end 116p, a distal end 116d and a tube lumen 1161 extending therethrough that connects the injection hub 118 to the core component 114c. In this embodiment, the secondary lumen extends from the core component 114c, along the flexible tube lumen 1161, through the injection hub 118, and terminates at the outlet port 118p. In the embodiment shown, the injection hub 118 further comprises an on-off valve 119, which is optional.

The flexible tubing 116 is intended to mechanically decouple the injection port 118i from the hub 114 of the cannula, which is advantageous, for example, in that it may further reduce the risk of unintentional shifting of the cannula away from a target ablation site during use. However, in other embodiments, a tube need not be included. For example, in some embodiments, an injection port comprising a mating feature that is configured to interface with an injection device is integrated into the cannula hub 114 and is positioned at a distal end of the secondary lumen.

As noted above, the cannula hub 114 includes a seal that is configured to form a watertight seal around a stylet shaft or an electrode shaft. As will be appreciated by those skilled in the art, the seal is useful in preventing fluid from flowing up the cannula and out of the distal end 114d of the cannula hub 114 (i.e., fluid backflow) when fluid is injected into the injection port 118i, even with the presence of an electrode within the cannula. Thus, the injection fluid is directed down the length of the cannula shaft 112 and out the cannula tip to the target site.

In the embodiment shown, the seal is a Tuohy-Borst seal which includes a proximal component 114tp, a distal component 114td, and an elastomeric gasket 114g positioned between the proximal component 114tp and the distal component 114td. The proximal component 114tp and the distal component 114td are adapted such that rotational movement of the proximal component 114tp relative to the distal component 114td causes the gasket 114g to either engage an inserted shaft in watertight seal or to disengage the inserted shaft, depending to the direction of rotation of the proximal component 114tp relative to the distal component 114td.

In the embodiment shown, the distal component 114td engages the core component 114c through a threaded arrangement. In other embodiments, however, the distal component 114td may be integrated into the core component 114c in a single unified part.

In the embodiment shown, the hub 114 is provided with a plurality of radially extended features 114e (e.g., wings, ridges, etc.) that assist in gripping and rotating the proximal component 114tp. Specifically, the hub 114 is provided with an adaptor cap 114a comprising extended features 114e in the form of wings 114e. The adaptor cap 114a is engageable with the proximal component 114tp by means of mating ridges or an adhesive in the embodiment shown. In other embodiments, the radially extended features that are provided on the adaptor cap 114a are directly provided on the proximal component 114tp itself and the adaptor cap 114a may be dispensed with.

In some embodiments, one or more radially extended features (e.g., wings, ridges, cut-outs, surface roughness, etc.) are provided at the distal end 114d of the cannula hub 114. One such embodiment is shown in FIG. 1C, which is a variation of FIG. 1B, in which wings 114e2 are provided at the distal end 114d of the cannula hub 114 (in addition to the wings 114e that are present on the adaptor cap 114a). For example, such wings 114e2 may be provided to assist in handling the cannula hub 114 and may also assist in rotating the proximal component 114tp relative to the distal component 114td.

It is noted that, although the seal in the embodiment described above is a Tuohy-Borst seal, alternative approaches may be employed to provide a water-tight seal between an inserted electrode shaft and the cannula hub and prevent fluid backflow. For example, in some embodiments, the electrode may be sealed to the cannula hub with a male-to-female luer taper in which the electrode hub mating surface of the cannula hub is lined with soft rubber-like material to improve the contact and grip in the electrode to cannula mate. Another alternative seal is the use of one or more O-rings to provide a seal between an inserted electrode shaft and the cannula hub. Yet another alternative seal design is based on a clamp having a soft elastomeric (e.g., silicone) sealing surface that is biased in the closed position, which presses down on the electrode shaft can be held open by the user when the electrode is being inserted or removed into the cannula. More generally, any suitable seal may be employed in the present disclosure. In this regard, because the injection port is decoupled from the electrode port in the present disclosure, this electrode insertion port now has the freedom to be of any desired seal design that is compatible with the electrodes and prevents fluid backflow.

As previously indicated and as shown in FIGS. 1A-1C, cannula hubs 114 in accordance with the present disclosure are configured to receive a stylet shaft 124 having a proximal end 124p and a distal end 124d and to interface with a stylet hub 122. A stylet can provide structural rigidity to the cannula during insertion into a patient. Moreover, by having a distal tip of the stylet shaft 124 terminate at the distal tip of the cannula shaft 112, coring of tissue during insertion of the cannula shaft 112 can be reduced or prevented.

In the embodiment shown, as with the cannula shaft 112, the stylet shaft 124 includes a bend 124b proximate a distal end 124d of the stylet shaft 124. Such a bend 124b may be used in embodiments like that shown where the cannula shaft 112 is provided with a side opening 1120. To avoid having the distal tip of the stylet shaft 124 protrude from the side opening 1120 during insertion, the bend in the cannula shaft 112 and the bend in the stylet shaft 124 are preferably oriented in the same direction. In addition, when the stylet shaft 124 is fully inserted into the cannula shaft 112, a longitudinal position of the bend 124b in the stylet shaft 124 preferably corresponds to a longitudinal position of the bend 112b in the cannula shaft 112. Moreover, the angle of the bend 124b in the stylet shaft 124 preferably corresponds to the angle of the bend 112b in the cannula shaft 112.

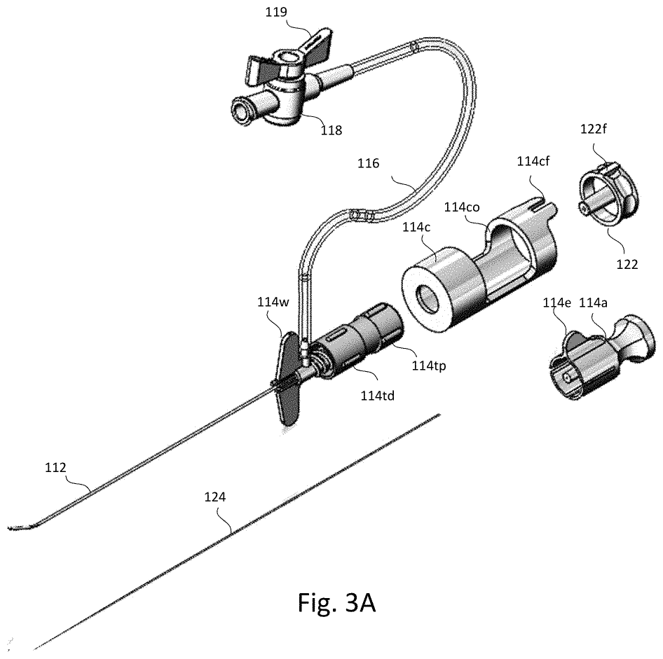

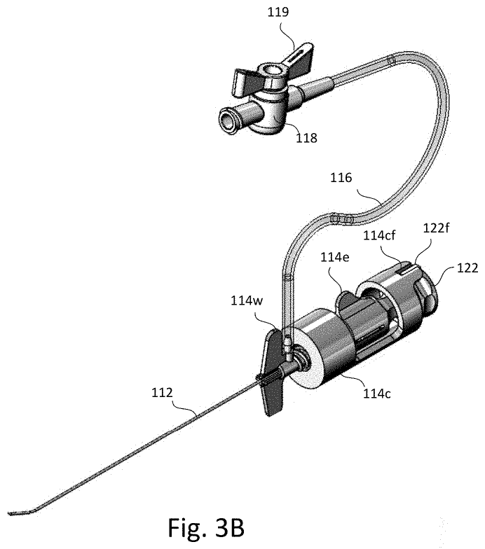

In embodiments such as those where a Tuohy-Borst adapter is used to form a seal, because the proximal end of the cannula hub is twisted relative to the distal end of the cannula hub, difficulties are encounter in orienting the bend 124b in the stylet shaft 124 such that it is oriented in the same direction as the bend 112b in the cannula shaft 112. To address this issue, in some embodiments, and with reference to FIGS. 3A-3B, a cannula hub 114 may be provided with an indexing component 114c that includes a first feature 114cf that extends past the portion of the cannula hub 114 that is twisted to form the seal (e.g., past the proximal component 114tp of the Tuohy-Borst adapter, and the Tuohy-Borst adapter cap 114a in the embodiment shown) and is that is configured to mate with a second feature 122f of a stylet hub 122, which allows the stylet shaft 124 to be placed at a predetermined rotational orientation relative to a rotational orientation of the cannula shaft 112.

In the embodiment shown the first feature 114cf is in the form of a slot and the second feature 122f is in the form of a ridge. Numerous other designs are possible, including designs where the first feature is in the form of a ridge and the second feature is in the form of a slot, among many others. The embodiment shown is designed to allows the stylet shaft 124 to be placed at a single predetermined rotational orientation relative to the rotational orientation of the cannula shaft 112. In other embodiments, the stylet shaft 124 may be placed at one of a plurality of rotational orientations relative to the rotational orientation of the cannula shaft 112. For example, a plurality of first features 114cf is in the form of slots may provide this function.

In the embodiment shown, the indexing component 114c is further provided with a side opening 114co which allows the operator to grasp and rotate the adaptor cap 114a, thereby engaging and disengaging an inserted shaft with the Tuohy-Borst seal. Also, although a single extended feature 114e in the form of a wing is illustrated, other embodiments such as ridges, etc. may be employed for this purpose as well.

Although not shown, analogous embodiments can be employed to allow a hub of an electrode shaft to be placed at one or more predetermined rotational orientations relative to a rotational orientation of the cannula shaft.

With regard to materials, in particular embodiments, the cannula shaft 112 may be formed from metallic materials including iron-chromium alloys, such as stainless steel, nickel-titanium alloys, such as nitinol, and nickel-chromium alloys, such as Inconel.

The various elements of the cannula hub (e.g., the core component 114c, the proximal component 114tp, the distal component 114td, the adaptor cap 114a, and the indexing component 114c), the injection hub 118, and the stylet hub 122 may be formed from any suitable material, with plastics, for example, acrylic polymers, polycarbonate, polypropylene, polycarbonate, acrylonitrile butadiene styrene (ABS), ABS/polycarbonate blends, polyethylene and nylon being beneficial in some embodiments.

In certain embodiments, the preceding components may be color coded, for example, to designate shaft length or needle gauge size.

Materials that may be used to form the Tuohy-Borst gasket 114g include elastomeric materials such as silicone, rubbers or nitrile.

Materials that may be used to form the flexible tube 116 include materials such as rubber, silicone, polyethylene terephthalate, or polyurethane, including thermoplastic polyurethanes such as Pellethane® thermoplastic polyurethanes.

Materials that may be used to form the stylet shaft 124 include, for example, polypropylene, polycarbonate, acrylonitrile butadiene styrene (ABS), ABS/polycarbonate blends and metal alloys, including stainless steel alloys.

Various additional aspects of the present disclosure pertain to methods of using the systems described herein, which include an RF ablation cannula, cannula stylet, and RF ablation electrode, to perform RF ablation in the peripheral nervous system for pain management.

In one exemplary embodiments, a procedure may be employed that includes the following steps: (1) injecting a local anesthetic on a skin surface near a cannula entry point, (2) inserting the cannula into a patient at a target location, (3) removing the cannula stylet and inserting the electrode into the cannula, (4) tightening the electrode seal (e.g., a Tuohy-Borst seal or other seal that is adapted to prevent fluid backflow) onto the electrode, (5) performing sensory and motor testing with the electrode inserted in order to verify cannula placement (adjusting placement as necessary), (6) attaching a syringe of local anesthetic to injection port of the cannula, (7) injecting local anesthetic at target location, (8) ablating tissue, and (9) removing electrode and cannula from patient.

As previously noted, a traditional RF ablation procedure includes the following steps: (1) injecting a local anesthetic on a skin surface near a cannula entry point, (2) inserting a cannula into patient to target location, (3) removing the cannula stylet and inserting an electrode into cannula, (4) performing sensory and motor testing with electrode inserted in order to verify cannula placement (adjusting placement as necessary), (5) removing the electrode from the cannula, (6) attaching a syringe of local anesthetic to the cannula, (7) injecting local anesthetic at the target location, (8) removing the syringe from the cannula, (9) re-inserting the electrode into the cannula, (10) ablating tissue, and (11) removing the electrode and the cannula from the patient.

It will be appreciated that, because the present disclosure employs an auxiliary injection port in addition to the insertion port for the electrode, steps (5), (8), and (9) of the traditional RF ablation procedure can be eliminated. Although the present disclosure has an added step of tighten the electrode seal onto the electrode to prevent fluid backflow, the present disclosure nonetheless simplifies the steps of the procedure overall, as well as cutting down on procedure time. Additionally, by reducing the number of times the electrode must be inserted into the cannula from two times to one time, the present disclosure decreases the risk of unintentional shifting of the cannula away from the target ablation site.

Variations, modifications, and other implementations of the present disclosure in addition to the various embodiments described herein will occur to those of ordinary skill in the art. Accordingly, the present disclosure is to be defined not by the preceding illustrative description but instead by the following claims.

1. An RF ablation cannula comprising:
a cannula shaft comprising a proximal end, a distal end, and a shaft lumen; and
a cannula hub comprising a proximal end, a distal end, a primary lumen having a first axis, a proximal end and a distal end, the primary lumen extending from the proximal end to the distal end of the cannula hub, wherein the distal end of the primary lumen terminates at a proximal end of the shaft lumen and the proximal end of the primary lumen terminates at an insertion port that is configured to receive an electrode shaft, a stylet shaft, or both, a secondary lumen having a proximal end and a distal end, the secondary lumen laterally branching from the primary lumen, wherein the proximal end of the secondary lumen terminates at the primary lumen and the distal end of the secondary lumen terminates at an injection port comprising a mating feature that is configured to interface with an injection device, and a seal that is configured to form a water-tight seal around a shaft that is inserted through the insertion port.

2. The RF ablation cannula of claim 1, wherein the seal comprises a proximal component, a distal component, and an elastomeric gasket between the proximal component and the distal component through which a portion of the primary lumen passes, wherein axial movement of the proximal component toward the distal component causes the elastomeric gasket to form the water-tight seal around the shaft that is inserted through the insertion port and wherein axial movement of the proximal component away from the distal component causes the elastomeric to release the shaft that is inserted through the insertion port.

3. The RF ablation cannula of claim 2, wherein rotation of the proximal component relative to the distal component in a first direction results in said axial movement of the proximal component toward the distal component and wherein rotation of the proximal component relative to the distal component in a second direction opposite the first direction results in said axial movement of the proximal component away from the distal component.

4. The RF ablation cannula of claim 2, wherein the cannula hub comprises a core component that contains a portion of the primary lumen and at least a portion of the secondary lumen, the secondary lumen laterally branching from the primary lumen within the core component, and wherein the distal component is either attached to the core component or is integrated with the core component as single unified component.

5. The RF ablation cannula of claim 2, wherein the seal comprises one or more radially extended features that assist in rotating the proximal component relative to the distal component.

6. The RF ablation cannula of claim 5, wherein the one or more radially extended features actuate the proximal component.

7. The RF ablation cannula of claim 6, wherein the one or more radially extended features are provided on the proximal component.

8. The RF ablation cannula of claim 6, wherein the one or more radially extended features are provided on an adaptor cap that attached to the proximal component.

9. The RF ablation cannula of claim 1, wherein the mating feature is a luer interface.

10. The RF ablation cannula of claim 1, wherein the cannula hub comprises a core component that contains a portion of the primary lumen and at least a portion of the secondary lumen, the secondary lumen laterally branching from the primary lumen within the core component.

11. The RF ablation cannula of claim 10, wherein the core component comprises the injection port.

12. The RF ablation cannula of claim 10, wherein the RF ablation cannula further comprises an injection hub comprising the injection port and a flexible tube connecting the injection hub to the core component, and wherein the secondary lumen extends from the core component, through the flexible tube, through the injection hub and terminates at the injection port.

13. The RF ablation cannula of claim 1, wherein the cannula shaft comprises a bend proximate the distal end of the cannula shaft and wherein the cannula shaft comprises a sidewall opening proximate the bend.

14. A system comprising:
an RF ablation cannula comprising: (a) a cannula shaft comprising a proximal end, a distal end, and a shaft lumen; and (b) a cannula hub comprising a proximal end, a distal end, a primary lumen having a first axis, a proximal end and a distal end, the primary lumen extending from the proximal end to the distal end of the cannula hub, wherein the distal end of the primary lumen terminates at a proximal end of the shaft lumen and the proximal end of the primary lumen terminates at an insertion port that is configured to receive an electrode shaft, a stylet shaft, or both, a secondary lumen having a proximal end and a distal end, the secondary lumen laterally branching from the primary lumen, wherein the proximal end of the secondary lumen terminates at the primary lumen and the distal end of the secondary lumen terminates at an injection port comprising a mating feature that is configured to interface with an injection device, and a seal that is configured to form a water-tight seal around a shaft that is inserted through the insertion port; and
a stylet comprising a stylet hub and a stylet shaft, the stylet shaft being insertable through the insertion port of the cannula hub and into the cannula shaft.

15. The system of claim 14, wherein the cannula shaft has a first bend, wherein the stylet shaft has a second bend, and wherein a longitudinal position of the first bend corresponds to a longitudinal position of the second bend when the stylet is fully inserted into the RF ablation cannula.

16. The system of claim 14, wherein the cannula hub comprises a first feature and wherein the stylet hub comprises a second feature that is configured to engage with the first feature when the stylet is fully inserted into the RF ablation cannula and when the stylet shaft is positioned at a specific predetermined rotational orientation relative to a rotational orientation of the cannula shaft.

17. The system of claim 16, wherein one of the first and second features is inserted into the other of the first and second features in order to engage the first and second features.

18. The system of claim 16, wherein the seal comprises a proximal component, a distal component, and an elastomeric gasket between the proximal component and the distal component, wherein the cannula hub comprises an indexing component rotationally fixed with the distal component while allowing rotation of the proximal component relative to the distal component, and wherein the indexing component comprises the first feature.

19. The system of claim 16, further comprising an RF ablation electrode having an electrode shaft.

20. A method of RF ablation comprising:
inserting an RF ablation cannula and stylet into a target location in a patient; the RF ablation cannula comprising: (a) a cannula shaft comprising a proximal end, a distal end, and a shaft lumen and (b) a cannula hub comprising a proximal end, a distal end, a primary lumen having a first axis, a proximal end and a distal end, the primary lumen extending from the proximal end to the distal end of the cannula hub, wherein the distal end of the primary lumen terminates at a proximal end of the shaft lumen and the proximal end of the primary lumen terminates at an insertion port that is configured to receive an electrode shaft, a stylet shaft, or both, a secondary lumen having a proximal end and a distal end, the secondary lumen laterally branching from the primary lumen, wherein the proximal end of the secondary lumen terminates at the primary lumen and the distal end of the secondary lumen terminates at an injection port comprising a mating feature that is configured to interface with an injection device, and a seal that is configured to form a water-tight seal around a shaft that is inserted through the insertion port; the stylet comprising a stylet hub and a stylet shaft, the stylet shaft being inserted through the insertion port of the cannula hub and into the cannula shaft;
removing the stylet;
inserting an RF ablation electrode into the RF ablation cannula through the insertion port of the cannula hub and into the cannula shaft;
tightening the seal onto the electrode shaft;
performing sensory and motor testing on the patient with the RF ablation electrode;
injecting local anesthetic through the injection port to the target location; and
ablating tissue with the RF ablation electrode.
